(19) United States
(12) Patent Application Publication
(10) Pub. No.: US 2013/0046264 A1
(43) Pub. Date: Feb. 21, 2013

(54) SKIN PATCH FOR ABSORBING TOXINS FROM THE BODY

(71) Applicant: Nurman SALIM, Shimada (JP)

(72) Inventor: Nurman SALIM, Shimada (JP)

(21) Appl. No.: 13/656,239

(22) Filed: Oct. 19, 2012

Related U.S. Application Data
(63) Continuation-in-part of application No. 12/267,731, filed on Nov. 10, 2008, now Pat. No. 8,317,762.

(51) Int. Cl. A61N 5/06; A61F 13/538

(52) U.S. Cl. 604/375; 607/88

(57) ABSTRACT

A skin patch for absorbing toxins from a body includes a first side and a second side. The second side includes a surface configured for absorbing and retaining the toxins. The surface includes a plurality of openings configured for enhancing transmission of far infrared ray emission into a skin when the second side is placed in contact with the skin. A mixture of natural ingredients includes wood vinegar, dextrin, and at least one additional ingredient selected from the group consisting of a mixture of natural ingredients, the mixture comprising wood vinegar, dextrin, and at least one additional ingredient selected from the group consisting of: bamboo vinegar, chitosan, loquat leaf, houttuynia cordata, vitamin C, tourmaline, vegetable fiber, sporopollenin, mushroom chitosan, peppermint, dandelion, blackhaw, fennel, licorice, capsaicin, agaricus, grapefruit, citrus, vaccinum berries, rubus berries, lavender, milk thistle, green tea, cinnamon, Japanese ginseng, Japanese seaweed, glucomannan, aloe vera, ginger, arnica, nattokinase, winter cherry, holy basil, eucalyptus, amethyst, diatomaceous earth, and zeolite.

### CROSS-REFERENCE TO RELATED APPLICATIONS

This application is a continuation-in-part of U.S. patent application Ser. No. 12/267,731, filed Nov. 10, 2008, and entitled Skin Patch for Absorbing Toxins from the Body, the entire contents of which are hereby incorporated by reference.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

The present invention relates generally to skin patches. More particularly, the invention relates to a skin patch that absorbs toxins from the body using infrared rays.

### BACKGROUND

Far infrared rays are thought to have the potential to be used to generate blood circulation in the body. In 1870, Dr. Pleasanton, a researcher in the United States, published research showing the relationship between light frequency and the rate of growth of cells and tissues, as well as their rate of cell division. Also, Dr. Neils Finsen, cured lesions and variola using red and infrared rays.

More recent research performed by Tiina Karu, M.D., of the Laser Technology Center in Russia, holds that this spectrum of light speeds up cellular metabolic processes, such as, but not limited to, stimulating the activity of mitochondria, and triggering enzyme activity as well as the healing, regeneration, and normalization of damaged cell tissue. Sunshine is known to emit far infrared rays. Electricity can also generate far infrared rays. A method of far infrared generation with electricity is disclosed in U.S. Pat. No. 6,610,082. However, the conversion of sunshine or electricity to generate far infrared rays may not be an efficient method for promoting blood circulation and other cellular processes in the body.

In view of the foregoing, there is a need for improved techniques for providing far infrared rays for use in promoting blood circulation and other cellular processes in the body that do not require sunshine or electricity.

### SUMMARY

To achieve the forgoing and other objects and in accordance with the purpose of the invention, a skin patch for absorbing toxins from the body is presented.

In one embodiment, a skin patch for absorbing toxins from a body is presented. The skin patch includes a first side having a heat reflective foil. A second side includes a non-woven rayon surface for absorbing the toxins. The rayon surface includes a plurality of openings for enhancing far infrared ray emission into a skin when the second side is placed in contact with the skin. A mixture of natural ingredients including vinegar, chitosan, loquat leaf, houttuynia cordata, vitamin C, tourmaline, vegetable fiber, and dextrin. The mixture is formulated to emit the far infrared emission, whereby blood circulation is promoted and perspiration and any toxins contained therein are absorbed by the rayon surface.

In another embodiment a skin patch for absorbing toxins from a body is presented. The skin patch includes means for reflecting heat, means for absorbing the toxins, means for enhancing far infrared ray emissions into a skin and means for emitting the far infrared ray emissions, whereby blood circulation is promoted and perspiration and any toxins contained therein are absorbed by the absorbing means.

Other features, advantages, and object of the present invention will become more apparent and be more readily understood from the following detailed description, which should be read in conjunction with the accompanying drawings.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

FIGS. 1A and 1B illustrate an exemplary toxin-absorbing patch, in accordance with an embodiment of the present invention. FIG. 1A shows the front of the patch, and FIG. 1B shows the back of the patch.

Unless otherwise indicated illustrations in the figures are not necessarily drawn to scale.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiment are not necessarily to be construed as preferred or advantageous over other embodiments. Moreover, the terms “embodiments of the invention”, “embodiments” or “invention” do not require that all embodiments of the invention include the discussed feature, advantage or mode of operation.

The present invention is best understood by reference to the detailed figures and description set forth herein. Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive. The present invention will now be described in detail with reference to embodiments thereof as illustrated in the accompanying drawings.

Preferred embodiments of the present invention provide a toxin-absorbing patch that is used to generate far infrared rays from natural ingredients without the help of electricity and sunshine. Exemplary purposes of preferred embodiments of the present invention are to help improve blood circulation and to reduce waste and toxins in the body. In a preferred embodiment, a toxin-absorbing patch comprises powdered herbal ingredients. The patch is applied to the sole of a foot and emits far infrared rays onto the foot that causes the foot to perspire during application. The warmth from the far infrared rays helps improve blood circulation, and the perspiration absorbed into the patch may contain toxic waste such as, but not limited to, heavy metals. In preferred embodiments, the patch is easy to apply and works in six to eight hours of time. This allows for use during bedtime. In preferred embodiments, adhesive tape or a bandage is used to secure the toxin absorbing patch onto the skin.

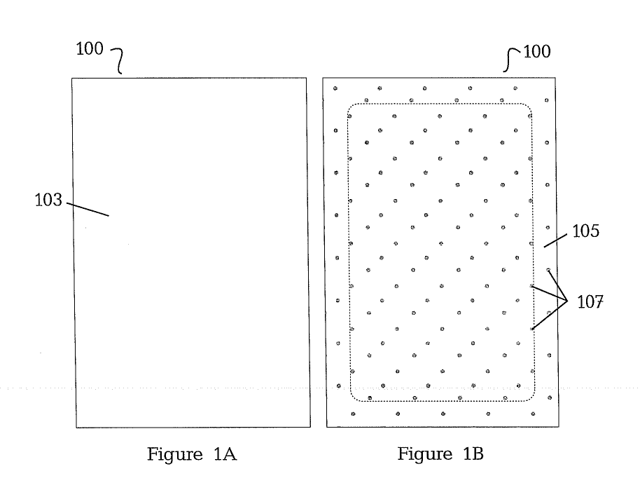

FIGS. 1A and 1B illustrate an exemplary toxin-absorbing patch 100, in accordance with an embodiment of the present invention. FIG. 1A shows the front of patch 100, and FIG. 1B shows the back of patch 100. The front section of patch 100 is made from thin-layered titanium foil 103, and the rear section of patch 100 is made from non-woven rayon 105. Foil 103 provides a very smooth, shiny and reflective surface, which can reflect heat. Adhesive tape is applied to foil 103 on the front section of patch 100 for application. Rayon 105 on the rear section of patch 100 is applied directly onto the skin during use. Rayon 105 comprises openings 107 that offer enhanced far infrared ray emission into the skin. Patch 100 preferably has a width of 60 mm and a length of 80 to 90 mm; however, patches in alternate embodiments may be various shapes and sizes. Patch 100 has large surface area so that it can cover a large section of the body. The weight of patch 100 is preferably five grams with an error level of 0.2 grams; however, patches in alternate embodiments may weigh more or less.

In other exemplary embodiments, the front section of patch 100 may be made from non-woven rayon, and the rear section of patch 100 may also be made from non-woven rayon. In yet other exemplary embodiments, the front section of patch 100 may be made from thin-layered titanium foil, and the rear section of patch 100 may also be made from thin-layered titanium foil. In yet other exemplary embodiments, the front and rear sections of patch 100 may be made from any suitable materials that enable patch 100 to function as described herein.

In the exemplary embodiment, powdered natural ingredients are sealed inside patch 100 between foil 103 and rayon 105. Patch 100 includes a sachet between foil 103 and rayon 105 that has a bag opening into which the natural ingredients such as, but not limited to, vinegar, chitosan, loquat leaf, houttuynia cordata, vitamin C, tourmaline, vegetable fiber, and dextrin may be inserted. In the present embodiment, the bag section of the sachet is large and can be made to contain five to eight grams of ingredients. This mixture of natural ingredients is formulated to emit heat, or far infrared rays. Table 1 lists an exemplary formula for the natural ingredients in a toxin-absorbing patch, according to an embodiment of the present invention. Those skilled in the art, in light of the present teachings, will readily recognize that various other types of ingredients and amounts of ingredients may be used in alternate embodiments.

In typical use of the present embodiment, patch 100 is applied externally on the surface of the skin. Patch 100 is attached to a bandage or adhesive tape before application to the skin. A user applies patch 100 to the skin by joining patch 100 to the bandage or adhesive tape with the edges of the bandage or adhesive tape extending beyond the edges of patch 100 and then attaching patch 100 with the adhesive tape or bandage to any location on the body that requires far infrared rays. Typically, the user applies patch 100 onto the bottom of a foot for six to eight hours. This allows for use during bedtime. During use, the foot produces sweat due to the heat coming from patch 100, and the sweat may include toxins such as, but not limited to, heavy metals, which are absorbed into patch 100. Therefore, a user that is suffering from heavy metal toxicity, for example, without limitation, a person with Autism, may benefit from the use of patch 100 because of the heavy metal reduction capability. The warmth from the far infrared rays also may help to improve blood circulation, and people suffering from blocked blood circulation may benefit from using patch 100. In the present embodiment, patch 100 fits virtually any part of the body, is easy to use, and generally eliminates the need for electrical devices and interference. Patch 100 is made of non-allergic material, and may therefore be used by people who have allergies.

Testing has shown that patches according to embodiments of the present invention may be used to emit heat when comprising a correct mixture of natural ingredients. Previously, only devices using electrical currents could be used to generate far infrared emission. Furthermore, an analysis of a new toxin-absorbing patch and a used toxin-absorbing patch was performed by Japan Food Research Laboratories on Dec. 2, 2005. The analysis tested for various heavy metals in the two patches including arsenic, lead, cadmium, mercury, tin, cobalt, and chromium. In the analysis only 0.01 parts per million (ppm) of cadmium was detected and none of the other metals were detected in the new patch while 0.55 ppm of lead, 0.03 ppm of cadmium, 0.09 ppm of cobalt, and 1.7 ppm of chromium were detected in the used patch. This analysis provides evidence that embodiments of the present invention may be used for the absorption of heavy metal from the body into the patch.

Additional exemplary embodiments of the patch 100 described herein may include formulations having a variety of ingredients in diverse combinations. The ingredients for the formulations may be selected from the non-limiting list of: loquat leaf (Eriobotrya japonica); dokudami (Houttuynia cordata); peppermint (Mentha piperita); dandelion (Taraxacum officinale); blackhaw (Viburnum prunifolium); fennel (Foeniculum vulgare); licorice (Glycyrrhiza glabra); agaricus; grapefruit (Citrus×paradisi); raspberry (Rubus); lavender (Lavandula); milk thistle (Silybum marianum); cinnamon (Cinnamomum); Japanese ginseng (Panax japonicus); Japanese seaweed (kaiso); konjac (Amorphophallus konjac); aloe vera; ginger (Zingiber officinale); arnica, which may be an arnica species not containing helenalin; winter cherry (ashwagandha or Withania somnifera); holy basil (tulsi or Ocimum tenuiflorum); wasabi (Wasabia japonica); eucalyptus; Vitamin C; wood vinegar; bamboo vinegar; vegetable fiber; glycyrrhizic acid, beta-glucans; sporopollenin; glucomannan; anethole, mushroom chitosan; chitosan; pectin, anthocyanins; zingerone; shogaol; gingerol; dextrin; capsaicin; beta-elemene; glutathione; Epigallocatechin gallate (EGCG) and L-Theanine; nattokinase; tourmaline; amethyst; diatomaceous earth; and zeolite.

In some exemplary embodiments of the formulations, certain active ingredients listed above may be extracted from a particular plant or naturally-occurring substance containing the active ingredient. For example, sporopollenin may be extracted from Chlorella vulgaris; capsaicin may be extracted from capsicum peppers; anthocyanins may be extracted from Vaccinum berries, Rubus berries, or the like; EGCG and L-Theanine may be extracted from green tea; dextrin may be extracted from cornstarch; glycyrrhizic acid may be extracted from licorice; beta-glucans may be extracted from agaricus; pectin may be extracted from grapefruit, citrus fruits, or any other suitable plant; anethole may be extracted from fennel; glucomannan may be extracted from konjac; zingerone, shogaol, and gingerol may be extracted from ginger; beta-elemene and glutathione may be extracted from holy basil or any other suitable plant, and so forth. However, such active ingredients may be extracted from any suitable plant species containing the ingredient and included in the formulation.

It should be appreciated that some exemplary embodiments of the formulations may include the above-described active ingredients as extracted from the plant matter containing the active ingredient. In other exemplary embodiments of the formulations, the active ingredients may be included in the formulations as part of the plant matter containing the active ingredients. It should further be appreciated that the amount of active ingredient in a particular plant may be an amount less than the total composition of the particular plant. For example, glycyrrhizic acid may be about 64% of licorice, by weight; beta-glucans may be about 99% of agaricus by weight; pectin may be about 33% of grapefruit by weight; EGCG may be about 80% of green tea by weight; L-theanine may be about 19% of green tea by weight; and glucomannan may be about 99% of konjac-mannan by weight. Therefore, where the below listed formulations recite a particular plant, the formulations should also be understood as encompassing any formulation containing any active ingredient of the plant, with the percentage by weight of the active ingredient in the formulation being substantially similar to the percentage by weight of the plant multiplied by the percentage by weight of the active ingredient contained in the plant. The formulations listed below should also be understood as equivalent to formulations including any substance, such as plant matter or the like, that includes the particular active ingredient.

Various embodiments of formulations for patch 100 may include the above-described ingredients in varying proportions. As shown in Table 1, a particular formulation may contain between 0% to 100%, by weight, of each of the above-described ingredients. It should be appreciated that the percentage by weight of each ingredient of the formulation may be adjusted as desired within the given weight-percent range.

TABLE 1

| Ingredient | Wt. % |
|---|---|
| Wood Vinegar | 0%-100% |
| Bamboo Vinegar | 0%-100% |
| Chitosan | 0%-100% |
| Loquat Leaf | 0%-100% |
| Houttuynia cordata | 0%-100% |
| Vitamin C | 0%-100% |
| Tourmaline | 0%-100% |
| Dextrin | 0%-100% |
| Vegetable fiber | 0%-100% |
| Sporopollenin | 0%-100% |
| Mushroom Chitosan | 0%-100% |
| Peppermint | 0%-100% |
| Dandelion | 0%-100% |
| Blackhaw | 0%-100% |
| Fennel | 0%-100% |
| Licorice | 0%-100% |
| Capsaicin | 0%-100% |
| Agaricus | 0%-100% |
| Grapefruit/citrus | 0%-100% |
| Vaccinum/Rubus berries | 0%-100% |
| Lavender | 0%-100% |
| Milk Thistle | 0%-100% |
| Green tea | 0%-100% |
| Cinnamon | 0%-100% |
| Wasabi | 0%-100% |
| Japanese ginseng | 0%-100% |
| Japanese seaweed | 0%-100% |
| Glucomannan | 0%-100% |
| Aloe Vera | 0%-100% |
| Ginger | 0%-100% |
| Arnica | 0%-100% |
| Nattokinase | 0%-100% |
| Winter cherry | 0%-100% |
| Holy basil | 0%-100% |
| Eucalyptus | 0%-100% |
| Amethyst | 0%-100% |
| Diatomaceous earth | 0%-100% |
| Zeolite | 0%-100% |

Exemplary embodiments of patch 100 may have total weights in the range between about 3 grams and about 50 grams. The embodiments of patch 100 may be provided within this range, for example, in 1 gram increments. Therefore, exemplary embodiments of patch 100 may include patches having a weight of 3 g, 4 g, 5 g, . . . , 48 g, 49 g, 50 g, inclusive of all intermediate sizes.

Additionally, patch 100 may be provided in any desired shape or size. Exemplary embodiments of patch 100 may have a length in the range between about 100 mm to about 2000 mm, and a width in the range between about 100 mm to about 2000 mm. Any sizes of patch 100 within these ranges may be provided, as a non-limiting example, 600 mm×800 mm, 600 mm×900 mm, 600 mm×1000 mm, 600 mm×1100 mm, 500 mm ×800 mm, and so forth. Patch 100 may be shaped as a circle, triangle, quadrilateral, or any other desired shape. In some exemplary embodiments, patch 100 may have a radius between about 100 mm to about 2000 mm. Therefore, any radii for patch 100 within these ranges may be provided, for example a radius of 100 mm, 200 mm, 300 mm, . . . , 1800 mm, 1900 mm, 2000 mm, inclusive of all intermediate sizes.

Tables 2-38 illustrate additional exemplary embodiments of formulations for patch 100. The ingredient proportions in the listed formulations should be considered exemplary and not limiting. It should also be appreciated that, for each composition, the percentage by weight of each ingredient may vary by approximately 50% of the indicated percentage by weight value for that ingredient. The formulations listed herein, as well as the percentage by weight values of the ingredients therein, may be applicable to embodiments of patch 100 having any desired weight or shape.

TABLE 2

| Ingredient | Wt. % |
|---|---|
| Wood Vinegar | 12.00% ± 6.00% |
| Bamboo Vinegar | 12.00% ± 6.00% |
| Chitosan | 0.80% ± 0.40% |
| Loquat Leaf | 0.80% ± 0.40% |
| Houttuynia cordata | 0.80% ± 0.40% |
| Vitamin C | 0.80% ± 0.40% |
| Tourmaline | 60.00% ± 30.00% |
| Vegetable Fiber | 4.00% ± 2.00% |
| Dextrin | 8.80% ± 4.40% |

TABLE 3

| Ingredient | Wt. % |
|---|---|
| Wood Vinegar | 26.00% ± 13.00% |
| Bamboo Vinegar | 15.00% ± 7.50% |
| Chitosan | 1.00% ± 0.50% |
| Loquat Leaf | 1.00% ± 0.50% |
| Houttuynia cordata | 5.00% ± 2.50% |
| Vitamin C | 2.00% ± 1.00% |
| Tourmaline | 30.00% ± 15.00% |
| Dextrin | 20.00% ± 10.00% |

TABLE 4

| Ingredient | Wt. % |
|---|---|
| Wood Vinegar | 15.00% ± 7.50% |
| Bamboo Vinegar | 15.00% ± 7.50% |
| Chitosan | 1.00% ± 0.50% |
| Loquat Leaf | 1.00% ± 0.50% |
| Houttuynia cordata | 1.00% ± 0.50% |
| Vitamin C | 1.00% ± 0.50% |
| Tourmaline | 50.00% ± 25.00% |
| Dextrin | 11.00% ± 5.50% |
| Vegetable fiber | 5.00% ± 2.50% |

TABLE 5

| Ingredient | Wt. % |
|---|---|
| Wood Vinegar | 11.76% ± 5.88% |
| Bamboo Vinegar | 7.06 ± 3.53%% |
| Loquat Leaf | 0.47% ± 0.235% |
| Houttuynia cordata | 0.47% ± 0.235% |
| Vitamin C | 0.47% ± 0.235% |
| Tourmaline | 70.59% ± 35.295% |
| Dextrin | 1.18% ± 0.59% |
| Vegetable fiber | 2.35% ± 1.175% |
| Sporopollenin | 5.18% ± 0.259% |
| Mushroom Chitosan | 0.47% ± 0.235% |

TABLE 6

| Ingredient | Wt. % |
|---|---|
| Wood Vinegar | 20.00% ± 10.00% |
| Tourmaline | 40.00% ± 20.00% |
| Dextrin | 4.00% ± 2.00% |
| Vegetable fiber | 4.00% ± 2.00% |
| Peppermint | 16.00% ± 8.00% |
| Dandelion | 4.00% ± 2.00% |
| Blackhaw | 4.00% ± 2.00% |
| Fennel | 4.00% ± 2.00% |
| Licorice | 4.00% ± 2.00% |

TABLE 7

| Ingredient | Wt. % |
|---|---|
| Wood Vinegar | 20.00% ± 10.00% |
| Tourmaline | 50.00% ± 25.00% |
| Dextrin | 2.00% ± 1.00% |
| Peppermint | 20.00% ± 10.00% |
| Dandelion | 2.00% ± 1.00% |
| Blackhaw | 2.00% ± 1.00% |
| Fennel | 2.00% ± 1.00% |
| Licorice | 2.00% ± 1.00% |

TABLE 8

| Ingredient | Wt. % |
|---|---|
| Wood Vinegar | 25.00% ± 12.50% |
| Bamboo Vinegar | 25.00% ± 12.50% |
| Chitosan | 0.50% ± 0.25% |
| Loquat Leaf | 5.00% ± 2.50% |
| Houttuynia cordata | 5.00% ± 2.50% |
| Vitamin C | 2.00% ± 1.00% |
| Tourmaline | 18.75% ± 9.375% |
| Dextrin | 12.50% ± 6.25% |
| Amethyst | 6.25% ± 3.125% |

TABLE 9

| Ingredient | Wt. % |
|---|---|
| Wood Vinegar | 23.75% ± 11.875% |
| Bamboo Vinegar | 56.25% ± 28.125% |
| Chitosan | 1.00% ± 0.50% |
| Loquat Leaf | 1.00% ± 0.50% |
| Houttuynia cordata | 2.00% ± 1.00% |
| Vitamin C | 1.00% ± 0.50% |
| Dextrin | 15.00% ± 7.50% |

TABLE 10

| Ingredient | Wt. % |
|---|---|
| Wood Vinegar | 25.00% ± 12.50% |
| Bamboo Vinegar | 56.25% ± 28.125% |
| Chitosan | 1.00% ± 0.50% |
| Loquat Leaf | 1.00% ± 0.50% |
| Houttuynia cordata | 2.00% ± 1.00% |
| Vitamin C | 1.00% ± 0.50% |
| Dextrin | 13.75% ± 6.875% |

TABLE 11

| Ingredient | Wt. % |
|---|---|
| Wood Vinegar | 15.00% ± 12.50% |
| Bamboo Vinegar | 15.00% ± 112.50% |
| Chitosan | 1.00% ± 0.50% |
| Loquat Leaf | 1.00% ± 0.50% |
| Houttuynia cordata | 1.50% ± 0.75% |
| Vitamin C | 1.00% ± 0.50% |
| Capsaicin | 2.50% ± 1.25% |
| Vegetable Fiber | 20.00% ± 10.00% |
| Dextrin | 43.00% ± 21.50% |

TABLE 12

| Ingredient | Wt. % |
|---|---|
| Wood Vinegar | 15.00% ± 7.50% |
| Bamboo Vinegar | 25.00% ± 12.50% |
| Chitosan | 1.00% ± 0.50% |
| Loquat Leaf | 1.00% ± 0.50% |
| Houttuynia cordata | 1.50% ± 0.75% |
| Vitamin C | 1.00% ± 0.50% |
| Capsaicin | 5.00% ± 2.50% |
| Vegetable Fiber | 20.00% ± 10.00% |
| Dextrin | 30.50% ± 15.25% |

TABLE 13

| Ingredient | Wt. % |
|---|---|
| Wood Vinegar | 16.00% ± 18.00% |
| Bamboo Vinegar | 20.00% ± 110.00% |
| Chitosan | 0.04% ± 10.02% |
| Loquat Leaf | 0.04% ± 10.02% |
| Houttuynia cordata | 0.06% ± 10.03% |
| Vitamin C | 0.80% ± 10.40% |
| Capsaicin | 1.00% ± 10.50% |
| Vegetable Fiber | 16.00% ± 18.00% |
| Dextrin | 24.40% ± 112.20% |

TABLE 14

| Ingredient | Wt. % |
|---|---|
| Wood Vinegar | 15.00% ± 7.50% |
| Bamboo Vinegar | 15.00% ± 7.50% |
| Tourmaline | 5.00% ± 2.50% |
| Agaricus | 5.00% ± 2.50% |
| Dextrin | 60.00% ± 30.00% |

TABLE 15

| Ingredient | Wt. % |
|---|---|
| Wood Vinegar | 12.00% ± 6.00% |
| Bamboo Vinegar | 12.00% ± 6.00% |
| Tourmaline | 20.00% ± 10.00% |
| Agaricus | 8.00% ± 4.00% |
| Dextrin | 48.00% ± 24.00% |

TABLE 16

| Ingredient | Wt. % |
|---|---|
| Wood Vinegar | 15.00% ± 7.50% |
| Bamboo Vinegar | 15.00% ± 7.50% |
| Vitamin C | 1.00% ± 0.50% |
| Vegetable Fiber | 20.00% ± 10.00% |
| Dextrin | 49.00% ± 24.50% |

TABLE 17

| Ingredient | Wt. % |
|---|---|
| Wood Vinegar | 16.00% ± 8.00% |
| Bamboo Vinegar | 16.00% ± 8.00% |
| Vitamin C | 4.80% ± 2.40% |
| Vegetable Fiber | 20.00% ± 10.00% |
| Dextrin | 43.20% ± 21.60% |

TABLE 18

| Ingredient | Wt. % |
|---|---|
| Wood Vinegar | 15.00% ± 7.50% |
| Bamboo Vinegar | 56.25% ± 28.125% |
| Loquat Leaf | 1.00% ± 0.50% |
| Houttuynia cordata | 2.00% ± 1.00% |
| Grapefruit/citrus | 2.00% ± 1.00% |
| Dextrin | 23.75% ± 11.875% |

TABLE 19

| Ingredient | Wt. % |
|---|---|
| Wood Vinegar | 16.00% ± 8.00% |
| Bamboo Vinegar | 45.00% ± 22.50% |
| Loquat Leaf | 2.00% ± 1.00% |
| Houttuynia cordata | 2.00% ± 1.00% |
| Grapefruit/citrus | 16.00% ± 8.00% |
| Dextrin | 19.00% ± 9.50% |

TABLE 20

| Ingredient | Wt. % |
|---|---|
| Wood Vinegar | 15.00% ± 7.50% |
| Bamboo Vinegar | 56.25% ± 28.125% |
| Loquat Leaf | 1.00% ± 0.50% |
| Houttuynia cordata | 2.00% ± 1.00% |
| Vaccinum/Rubus berries | 2.00% ± 1.00% |
| Dextrin | 23.75% ± 11.275% |

TABLE 21

| Ingredient | Wt. % |
|---|---|
| Wood Vinegar | 16.00% ± 8.00% |
| Bamboo Vinegar | 45.00% ± 22.50% |
| Loquat Leaf | 2.00% ± 1.00% |
| Houttuynia cordata | 2.00% ± 1.00% |
| Vaccinum/Rubus berries | 16.00% ± 8.00% |
| Dextrin | 19.00% ± 9.50% |

TABLE 22

| Ingredient | Wt. % |
|---|---|
| Wood Vinegar | 15.00% ± 7.50% |
| Bamboo Vinegar | 56.25% ± 28.125% |
| Loquat Leaf | 1.00% ± 0.50% |
| Houttuynia cordata | 2.00% ± 1.00% |
| Lavender | 2.00% ± 1.00% |
| Dextrin | 23.75% ± 11.275% |

TABLE 23

| Ingredient | Wt. % |
|---|---|
| Wood Vinegar | 16.00% ± 8.00% |
| Bamboo Vinegar | 45.00% ± 22.50% |
| Loquat Leaf | 0.80% ± 0.40% |
| Houttuynia cordata | 1.60% ± 0.80% |
| Tourmaline | 5.60% ± 2.80% |
| Lavender | 20.00% ± 10.00% |
| Dextrin | 11.00% ± 5.50% |

TABLE 24

| Ingredient | Wt. % |
|---|---|
| Wood Vinegar | 25.00% ± 12.50% |
| Bamboo Vinegar | 25.00% ± 12.50% |
| Loquat Leaf | 1.50% ± 0.75% |
| Houttuynia cordata | 1.00% ± 0.50% |
| Tourmaline | 12.50% ± 6.25% |
| Grapefruit/citrus | 25.00% ± 12.50% |
| Dextrin | 10.00% ± 5.00% |

TABLE 25

| Ingredient | Wt. % |
|---|---|
| Wood Vinegar | 20.00% ± 10.00% |
| Bamboo Vinegar | 20.00% ± 10.00% |
| Loquat Leaf | 1.20% ± 0.60% |
| Houttuynia cordata | 0.80% ± 0.40% |
| Tourmaline | 20.00% ± 10.00% |
| Grapefruit/citrus | 30.00% ± 15.00% |
| Dextrin | 8.00% ± 4.00% |

TABLE 26

| Ingredient | Wt. % |
|---|---|
| Wood Vinegar | 25.00% ± 12.50% |
| Bamboo Vinegar | 12.50% ± 6.25% |
| Loquat Leaf | 5.00% ± 2.50% |
| Houttuynia cordata | 5.00% ± 2.50% |
| Tourmaline | 12.50% ± 6.25% |
| Vegetable Fiber | 5.00% ± 2.50% |
| Capsaicin | 5.00% ± 2.50% |
| Milk thistle | 10.00% ± 5.00% |
| Dextrin | 20.00% ± 10.00% |

TABLE 27

| Ingredient | Wt. % |
|---|---|
| Wood Vinegar | 15.00% ± 7.50% |
| Bamboo Vinegar | 15.00% ± 7.50% |
| Loquat Leaf | 0.50% ± 0.25% |
| Houttuynia cordata | 0.50% ± 0.25% |
| Vitamin C | 0.50% ± 0.25% |
| Tourmaline | 12.50% ± 6.25% |
| Vegetable Fiber | 1.00% ± 0.50% |
| Green tea | 50.00% ± 25.00% |
| Dextrin | 5.00% ± 2.50% |

TABLE 28

| Ingredient | Wt. % |
|---|---|
| Wood Vinegar | 12.00% ± 6.00% |
| Bamboo Vinegar | 12.00% ± 6.00% |
| Loquat Leaf | 0.40% ± 0.20% |
| Houttuynia cordata | 0.40% ± 0.20% |
| Vitamin C | 0.40% ± 0.20% |
| Tourmaline | 20.00% ± 10.00% |
| Vegetable Fiber | 0.80% ± 0.40% |
| Green tea | 50.00% ± 25.00% |
| Dextrin | 4.00% ± 2.00% |

TABLE 29

| Ingredient | Wt. % |
|---|---|
| Wood Vinegar | 15.00% ± 7.50% |
| Bamboo Vinegar | 15.00% ± 7.50% |
| Chitosan | 0.50% ± 0.25% |
| Loquat Leaf | 0.50% ± 0.25% |
| Houttuynia cordata | 0.50% ± 0.25% |
| Tourmaline | 37.50% ± 18.75% |
| Vegetable Fiber | 0.50% ± 0.25% |
| Cinnamon | 10.00% ± 5.00% |
| Japanese ginseng | 5.00% ± 2.50% |
| Japanese seaweed | 5.00% ± 2.50% |
| Glucomannan | 5.00% ± 2.50% |
| Dextrin | 5.50% ± 2.50% |

TABLE 30

| Ingredient | Wt. % |
|---|---|
| Wood Vinegar | 12.00% ± 6.00% |
| Bamboo Vinegar | 12.00% ± 6.00% |
| Chitosan | 0.40% ± 0.20% |
| Loquat Leaf | 0.40% ± 0.20% |
| Houttuynia cordata | 0.40% ± 0.20% |
| Tourmaline | 50.00% ± 25.00% |
| Vegetable Fiber | 0.40% ± 0.20% |
| Cinnamon | 8.00% ± 4.00% |
| Japanese ginseng | 4.00% ± 2.00% |
| Japanese seaweed | 4.00% ± 2.00% |
| Glucomannan | 4.00% ± 2.00% |
| Dextrin | 4.40% ± 2.00% |

TABLE 31

| Ingredient | Wt. % |
|---|---|
| Wood Vinegar | 12.00% ± 6.00% |
| Bamboo Vinegar | 8.00% ± 4.00% |
| Houttuynia cordata | 4.00% ± 2.00% |
| Vitamin C | 0.80% ± 0.40% |
| Tourmaline | 20.00% ± 10.00% |
| Vegetable Fiber | 2.00% ± 1.00% |
| Diatomaceous earth | 20.00% ± 10.00% |
| Aloe Vera | 2.04% ± 1.02% |
| Ginger | 1.98% ± 0.99% |
| Arnica | 1.98% ± 0.99% |
| Zeolite | 20.00% ± 10.00% |
| Dextrin | 7.20% ± 3.60% |

TABLE 32

| Ingredient | Wt. % |
|---|---|
| Wood Vinegar | 11.76% ± 5.88% |
| Bamboo Vinegar | 11.76% ± 5.88% |
| Loquat Leaf | 0.47% ± 0.235% |
| Houttuynia cordata | 0.47% ± 0.235% |
| Vitamin C | 0.94% ± 0.47% |
| Tourmaline | 35.29% ± 17.645% |
| Vegetable Fiber | 2.35% ± 1.175% |
| Mushroom chitosan | 0.47% ± 0.235% |
| Nattokinase | 11.76% ± 5.88% |
| Winter cherry | 11.76% ± 5.88% |
| Holy basil | 11.76% ± 5.88% |
| Dextrin | 1.18% ± 0.59% |

TABLE 33

| Ingredient | Wt. % |
|---|---|
| Wood Vinegar | 8.00% ± 4.00% |
| Bamboo Vinegar | 12.00% ± 6.00% |
| Chitosan | 16.00% ± 8.00% |
| Tourmaline | 30.00% ± 15.00% |
| Agaricus | 4.00% ± 2.00% |
| Eucalyptus | 10.00% ± 5.00% |
| Dextrin | 20.00% ± 10.00% |

TABLE 34

| Ingredient | Wt. % |
|---|---|
| Wood Vinegar | 7.06% ± 3.53% |
| Bamboo Vinegar | 7.06% ± 3.53% |
| Loquat Leaf | 0.24% ± 0.12% |
| Houttuynia cordata | 0.24% ± 0.12% |
| Tourmaline | 70.59% ± 35.295% |
| Sporopollenin | 2.94% ± 1.47% |
| Amethyst | 2.94% ± 1.47% |
| Agaricus | 2.94% ± 1.47% |
| Zeolite | 2.94% ± 1.47% |
| Dextrin | 0.26% ± 0.13% |

TABLE 35

| Ingredient | Wt. % |
|---|---|
| Wood Vinegar | 11.76% ± 5.88% |
| Bamboo Vinegar | 7.06% ± 3.53% |
| Loquat Leaf | 0.24% ± 0.12% |
| Houttuynia cordata | 0.24% ± 0.12% |
| Vitamin C | 0.24% ± 0.12% |
| Tourmaline | 70.59% ± 35.295% |
| Vegetable fiber | 1.18 ± 0.59% |
| Sporopollenin | 7.06% ± 3.53% |
| Mushroom chitosan | 0.47% ± 0.235% |
| Dextrin | 0.18% ± 0.09% |

TABLE 36

| Ingredient | Wt. % |
|---|---|
| Wood Vinegar | 10.00% ± 5.00% |
| Bamboo Vinegar | 6.00% ± 3.00% |
| Loquat Leaf | 0.20% ± 0.10% |
| Houttuynia cordata | 0.20% ± 0.10% |
| Vitamin C | 0.20% ± 0.10% |
| Tourmaline | 70.00% ± 35.00% |
| Vegetable fiber | 2.00 ± 1.00% |
| Sporopollenin | 10.00% ± 5.00% |
| Mushroom chitosan | 0.40% ± 0.20% |
| Dextrin | 1.00% ± 0.50% |

TABLE 37

| Ingredient | Wt. % |
|---|---|
| Wood Vinegar | 9.09% ± 4.545% |
| Bamboo Vinegar | 5.45% ± 2.725% |
| Loquat Leaf | 0.18% ± 0.09% |
| Houttuynia cordata | 0.18% ± 0.09% |
| Vitamin C | 0.18% ± 0.09% |
| Tourmaline | 72.73% ± 36.365% |
| Vegetable fiber | 1.82 ± 0.91% |
| Sporopollenin | 9.09% ± 4.545% |
| Mushroom chitosan | 0.36% ± 0.18% |
| Dextrin | 0.91% ± 0.455% |

TABLE 38

| Ingredient | Wt. % |
|---|---|
| Wood Vinegar | 20.00% ± 10.00% |
| Bamboo Vinegar | 45.00% ± 22.50% |
| Chitosan | 0.80% ± 0.40 |
| Loquat Leaf | 0.80% ± 0.40% |
| Houttuynia cordata | 1.60% ± 0.80% |
| Wasabi | 20.00% ± 10.00% |
| Vegetable fiber | 2.00 ± 1.00% |
| Dextrin | 11.00% ± 5.50% |

Having fully described at least one embodiment of the present invention, other equivalent or alternative methods of providing toxin absorption and improving blood circulation using far infrared rays according to the present invention will be apparent to those skilled in the art. The invention has been described above by way of illustration, and the specific embodiments disclosed are not intended to limit the invention to the particular forms disclosed. For example, the particular implementation of the patch may vary depending upon the intended location of application. The patches described in the foregoing were directed to implementations typically applied to the bottom of the foot; however, similar techniques are to provide patches that are shaped to specifically fit on various parts of the body. For example, without limitation, large sheets may be used on the back or the back of the legs, curved patches may be used on the underarms, and small, round patches may be used on the hands. Implementations of the present invention that are applied to various areas of the body are contemplated as within the scope of the present invention. The invention is thus to cover all modifications, equivalents, and alternatives falling within the spirit and scope of the following claims.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A skin patch for absorbing toxins from a body, comprising:
a first side;
a second side comprising a surface configured for absorbing and retaining the toxins, the surface comprising a plurality of openings configured for enhancing transmission of far infrared ray emission into a skin when the second side is placed in contact with the skin; and
a mixture of natural ingredients, the mixture comprising wood vinegar, dextrin, and at least one additional ingredient selected from the group consisting of:
bamboo vinegar, chitosan, loquat leaf, houttuynia cordata, vitamin C, tourmaline, vegetable fiber, sporopollenin, mushroom chitosan, peppermint, dandelion, blackhaw, fennel, licorice, capsaicin, agaricus, grapefruit, citrus, vaccinum, berries, rubus berries, lavender, milk thistle, green tea, cinnamon, wasabi, Japanese ginseng, Japanese seaweed, glucomannan, aloe vera, ginger, arnica, nattokinase, winter cherry, holy basil, eucalyptus, amethyst, diatomaceous earth, and zeolite.

2. The skin patch of claim 1, wherein the first side is formed from one of a heat-reflective foil or a non-woven rayon surface.

3. The skin patch of claim 1, wherein the second side is formed from one of a heat-reflective foil or a non-woven rayon surface.

4. The skin patch of claim 1, wherein the natural ingredients are sealed between the first side and the second side.

5. The skin patch of claim 1, wherein the skin patch is operable to be joined to the skin with an adhesive.

6. The skin patch of claim 1, wherein the skin patch is operable to be joined to the skin of a bottom of a foot.

7. A skin patch for absorbing toxins from a body, comprising:
a first side;
a second side comprising a surface configured for absorbing and retaining the toxins, the surface comprising a plurality of openings configured for enhancing transmission of far infrared ray emission into a skin when the second side is placed in contact with the skin; and
a mixture of natural ingredients, the mixture comprising wood vinegar, dextrin, and at least one additional ingredient selected from the group consisting of:
bamboo vinegar, chitosan, loquat leaf, houttuynia cordata, vitamin C, tourmaline, vegetable fiber, sporopollenin, mushroom chitosan, peppermint, dandelion, blackhaw, anethole, glycyrrhizic acid, capsaicin, beta-glucans, pectin, anthocyanins, lavender, milk thistle, EGCG and L-theanine, cinnamon, wasabi, Japanese ginseng, Japanese seaweed, glucomannan, aloe vera, zingerone, shogaol, gingerol, arnica, nattokinase, winter cherry, beta-elemene, glutathione, eucalyptus, amethyst, diatomaceous earth, and zeolite.

8. The skin patch of claim 7, wherein the first side is formed from one of a heat-reflective foil or a non-woven rayon surface.

9. The skin patch of claim 7, wherein the second side is formed from one of a heat-reflective foil or a non-woven rayon surface.

10. The skin patch of claim 7, wherein the natural ingredients are sealed between the first side and the second side.

11. The skin patch of claim 7, wherein the skin patch is operable to be joined to the skin with an adhesive.

12. The skin patch of claim 7, wherein the skin patch is operable to be joined to the skin of a bottom of a foot.

13. A skin patch for absorbing toxins from a body, comprising:
means for reflecting heat from far infrared ray emissions;
means for absorbing and retaining the toxins;
means for enhancing transmission of far infrared ray emissions into a skin; and
means for emitting said far infrared ray emissions, whereby blood circulation is promoted and perspiration and any toxins contained therein are absorbed by said absorbing means;
wherein the skin patch contains a mixture of natural ingredients, the mixture comprising wood vinegar, dextrin, and at least one additional ingredient selected from the group consisting of:
bamboo vinegar, chitosan, loquat leaf, houttuynia cordata, vitamin C, tourmaline, vegetable fiber, sporopollenin, mushroom chitosan, peppermint, dandelion, blackhaw, fennel, licorice, capsaicin, agaricus, grapefruit, citrus, vaccinum berries, rubus berries, lavender, milk thistle, green tea, cinnamon, wasabi, Japanese ginseng, Japanese seaweed, glucomannan, aloe vera, ginger, arnica, nattokinase, winter cherry, holy basil, eucalyptus, amethyst, diatomaceous earth, and zeolite.

14. The skin patch of claim 13, further comprising means for joining the skin patch to the skin.
